(19) United States
(12) Patent Application Publication
(10) Pub. No.: US 2012/0165647 A1
(43) Pub. Date: Jun. 28, 2012

(54) APPARATUS FOR MEASURING SIZES OF HUMAN BODY USING MILLIMETER WAVES

(75) Inventors: Min-Soo Kang, Daejeon (KR); Kwang-Seon Kim, Daejeon (KR); Bong-Su Kim, Daejeon (KR); Woo-Jin Byun, Daejeon (KR)

(73) Assignee: Electronics and Telecommunications Research Institute, Daejeon (KR)

(21) Appl. No.: 13/330,154

(22) Filed: Dec. 19, 2011

(30) Foreign Application Priority Data
Dec. 23, 2010 (KR) 10-2010-0133813

(51) Int. Cl. A61B 6/00; G01S 13/00

(52) U.S. Cl. 600/407; 342/82

(57) ABSTRACT

Disclosed is an apparatus for measuring sizes of a human body, such as the length of an arm, the size of a waist and the width of a shoulder, in the state that a person does not take off its clothes, using millimeter waves. The apparatus includes a plurality of millimeter-wave transmission/reception units, a rotation unit and a digital signal processing unit. The plurality of millimeter-wave transmission/reception units are arranged in a circular shape around an object to be measured, transmit a millimeter wave to the object to be measured, and receive a signal reflected or scattered by the object to be measured. The rotation unit rotates a circular structure in which the plurality of millimeter-wave transmission/reception units are arranged. The digital signal processing unit analyzes the object to be measured using size and phase information of the signal received by the plurality of millimeter-wave transmission/reception units.

### CROSS-REFERENCE TO RELATED APPLICATIONS

The present application claims priority of Korean Patent Application No. 10-2010-0133813, filed on Dec. 23, 2010, which is incorporated herein by reference in its entirety.

### BACKGROUND OF THE INVENTION

1. Field of the Invention

Exemplary embodiments of the present invention relate to an apparatus for measuring sizes of a human body, such as the length of an arm, the size of a waist and the width of a shoulder, in the state that a person does not take off its clothes, using millimeter waves.

2. Description of Related Art

In the fashion industry, clothes were produced in large quantities, but are currently produced suitable for individual tastes in small quantities. That is, the concept of clothes is changing. Such a change reflects desires of people who intend not to purchase typical clothes but to purchase clothes suitable for their own tastes. Since the change in the fashion industry enables clothes suitable for individual tastes to be produced one by one through measurement of sizes for each individual, it is possible to make sales promotion through reduction of production period and to ensure price competitiveness through reduction of production cost.

A three-dimensional (3D) apparatus for measuring sizes of a human body is required to produce clothes suitable for individual tastes. Generally, the 3D apparatus uses a method using laser or white light. The method using the laser is a method in which a laser transmitter irradiates a laser beam, and a laser receiver positioned opposite to the laser transmitter decides the presence of reception of the laser beam, thereby extracting a 3D image. The method using the white light is a method of extracting a 3D image by casting a shadow onto a human body using light and then identifying curvature of the shadow.

The method using the laser and the method using the white light are all performed using light. However, since the light does not pass through clothes, the measurement of sizes of a human body should be performed in the state that a person takes off its clothes so as to measure precise sizes of the human body.

### SUMMARY OF THE INVENTION

An embodiment of the present invention is directed to an apparatus for measuring sizes of a human body using millimeter waves, which can measure the sizes of the human body even in the state that a person puts on simple clothes, using a characteristic in which a signal of a millimeter-wave signal does not pass through the human body but passes through the clothes.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

In accordance with an embodiment of the present invention, a measuring apparatus using millimeter waves includes a plurality of millimeter-wave transmission/reception units configured to be arranged in a circular shape around an object to be measured, transmit a millimeter wave to the object to be measured, and receive a signal reflected or scattered by the object to be measured; a rotation unit configured to rotate a circular structure in which the plurality of millimeter-wave transmission/reception units are arranged; and a digital signal processing unit configured to analyze the object to be measured using size and phase information of the signal received by the plurality of millimeter-wave transmission/reception units. In the apparatus, in one measurement cycle in which any one of the plurality of millimeter-wave transmission/reception units operates in a transmission mode and all the millimeter-wave transmission/reception units operate in a reception mode, the measurement may be sequentially performed until when all the millimeter-wave transmission/reception units operate one by one in the transmission mode.

When the one measurement cycle is completed, the circular structure may be rotated to a predetermined angle by the rotation unit, and another measurement cycle may be then performed.

The millimeter-wave transmission/reception unit may include a control unit configured to control the transmission and reception modes, control the generation of the millimeter wave, and provide a measured result to the digital signal processing unit; a millimeter-wave signal generation unit configured to generate a millimeter-wave signal under a control of the control unit; a transmission antenna configured to transmit, to the object to be measured, the millimeter-wave signal generated by the millimeter-wave signal generation unit; a reception antenna configured to receive the signal reflected or scattered by the object to be measured; and a reception unit configured to process the signal inputted from the reception antenna and provide the processed signal to the control unit.

The millimeter-wave transmission/reception unit may further include an image photographing unit configured to photograph an image of the object to be measured; and an image signal conversion unit configured to process the image photographed by the image photographing unit.

The digital signal processing unit may correct the measured result of the object to be measured based on the photographed image processed by the image signal conversion unit.

The reception antenna may be configured with two or more reception antennas so as to improve reception performance.

When the measurement in directions of 360 degrees is completed through the millimeter-wave transmission/reception units, the digital signal processing unit may estimate sizes of a human body of the object to be measured using an algorithm for solving an inverse scattering equation, based on the measured result.

The apparatus may further include a support platform configured to support the object to be measured.

The apparatus may further include an auxiliary handle disposed near the support platform so as to prevent a movement of the object to be measured.

The apparatus may further include a plurality of landmarks made of a material totally reflecting or absorbing a millimeter wave of the signal transmitted by the millimeter-wave transmission/reception unit and attached to the object to be measured as measurement reference points.

### BRIEF DESCRIPTION OF THE DRAWINGS

FIG. 1 illustrates an entire configuration of an apparatus for measuring sizes of a human body using millimeter waves in accordance with an embodiment of the present invention.

FIG. 2 is a block configuration diagram illustrating an embodiment of a millimeter-wave transmission/reception unit in accordance with the present invention.

FIG. 3 is a block configuration diagram illustrating another embodiment of the millimeter-wave transmission/reception unit in accordance with the present invention.

FIG. 4 illustrates an apparatus for measuring sizes of a human body using millimeter waves in accordance with another embodiment of the present invention.

FIG. 5 illustrates a method for measuring sizes of a human body using landmarks in accordance with an embodiment of the present invention.

### DESCRIPTION OF SPECIFIC EMBODIMENTS

Exemplary embodiments of the present invention will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. Throughout the disclosure, like reference numerals refer to like parts throughout the various figures and embodiments of the present invention.

Millimeter waves are electromagnetic waves with wavelengths corresponding to a frequency ranging from 30 to 300 GHz in a free space. Millimeter-wave bands have a superior straight characteristic to microwave bands, and are divided into a shadow band and a clear band depending on the transmission characteristic. Here, the shadow band is absorbed well in water molecules or oxygen molecules in the air, and the clear band has relatively less attenuation. Studies on applications using the attenuation of the millimeter waves have been conducted in various fields such as communication, medical services, traffic, imaging and military systems. Accordingly, millimeter-wave imaging techniques for sensing noise power using millimeter waves radiated from objects in a natural state have recently been developed as applications in security and medical fields.

An apparatus for measuring sizes of a human body in accordance with embodiments of the present invention can measure the sizes of the human body even in the state that a user to be measured puts on simple clothes, using a characteristic in which a signal of a millimeter-wave signal does not pass through the human body but passes through the clothes.

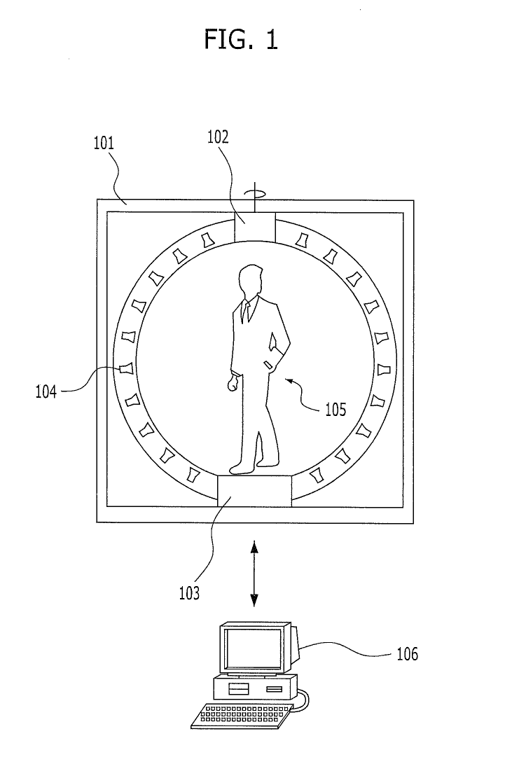

FIG. 1 illustrates an entire configuration of an apparatus for measuring sizes of a human body using millimeter waves in accordance with an embodiment of the present invention.

The apparatus in accordance with the embodiment of the present invention includes a plurality of millimeter-wave transmission/reception units 104 for transmitting/receiving millimeter-wave signals. The plurality of millimeter-wave transmission/reception units 104 are arranged in a circular structure. A motor for rotating the circular structure having the plurality of millimeter-wave transmission/reception units 104 arranged therein and a motor control unit 102 are provided to a frame 101. That is, the motor (not shown) is supported by the frame 101, and the circular structure connected to a rotary shaft of the motor is rotated in a predetermined direction under the control of the motor control unit 102.

The apparatus in accordance with the embodiment of the present invention further includes a support platform 103. The apparatus may further include a digital signal processing unit 106 including a measurement algorithm for receiving reception signals and estimating sizes of a human body using amplitude and size information of the reception signals. Here, the reception signals are transmitted by the plurality of millimeter-wave transmission/reception units 104 and then reflected by a person that is an object to be measured.

An operation of the apparatus in accordance with the embodiment of the present invention will be described as follows.

Any one of the plurality of millimeter-wave transmission/reception units 104 is selected, and a specific transmission signal of which amplitude and size information recognized by the selected millimeter-wave transmission/reception unit is applied to an object 105 to be measured. The transmission signal applied as described above is reflected and scattered by the object 105 to be measured.

The reflected and scattered signal is again received by a reception unit in each of the plurality of millimeter-wave transmission/reception units 104. That is, the transmission signal is applied from one millimeter-wave transmission/reception unit, and the reception signal are received by all the reception units in the plurality of millimeter-wave transmission/reception units 104. Information on the amplitudes and phases of the transmission and reception signals is transmitted to the digital signal processing unit 106.

The millimeter-wave transmission and reception processes are sequentially performed by all the millimeter-wave transmission/reception units. That is, the process is sequentially and repeatedly performed, in which any one selected as a transmission side from the plurality of millimeter-wave transmission/reception units transmits a millimeter wave and a signal reflected and scattered from the object to be measured are received by all the millimeter transmission/reception units.

If the millimeter-wave transmission and reception processes are completed in all the millimeter transmission/reception units, the circular structure is again rotated at a predetermined interval under the control of the motor control unit 102, and the millimeter-wave transmission and reception processes are again repeated. Through the aforementioned processes, basic data for measuring the sizes of the human body of the object 105 to be measured are collected.

In the spherical coordinate system, a single transmission signal is temporally radiated at a predetermined angle (the angle is determined by the interval between the millimeter-wave transmission/reception units) in directions Theta with respect to a same radius (r) and a specific angle Pi, and accordingly, values of the radiated signal reflected and scattered from the object 105 to be measured are all received in all the directions Theta. If the measurement is performed at the predetermined angle in all directions of 360 degrees with respect to the direction Pi, 3D basic data for measuring the sizes of the human body of the object 105 to be measured can be obtained. Information on the sizes of the human body of the object 105 to be measured is obtained using an algorithm for solving an inverse scattering equation, based the basic data.

That is, the digital signal processing unit 106 stores data measured in each measurement cycle. If the measurement is completed in all the measurement cycles, the digital signal processing unit 106 obtains the information on the sizes of the human body using the algorithm for solving the inverse scattering equation, based on the stored data.

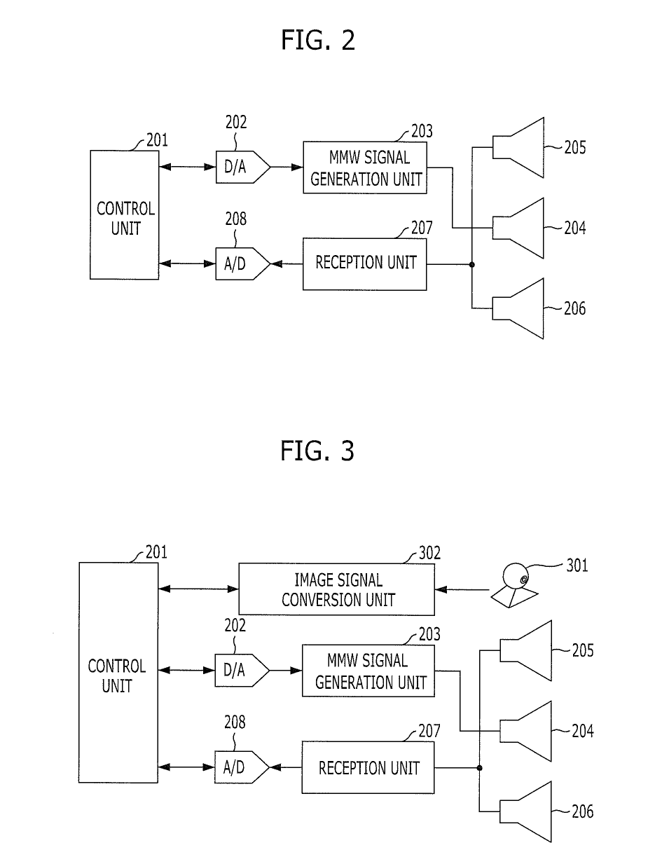

FIG. 2 is a block configuration diagram illustrating an embodiment of a millimeter-wave transmission/reception unit in accordance with the present invention.

The millimeter-wave transmission/reception unit includes a control unit 201, a digital/analog (D/A) conversion unit 202, a millimeter-wave band (MMW) signal generation unit 203, one transmission antenna 204, one or more reception antennas 205 and 206, a reception unit 207, an analog/digital (A/D) conversion unit 208, and a control unit 201. The control unit 201 controls a digital signal in communication with the digital signal processing unit 106, and processes a reception signal inputted from the A/D conversion unit 208 and then transmits the processed reception signal to the digital signal processing unit 106. The D/A conversion unit 202 converts the digital signal inputted from the control unit 201. The MMW signal generation unit 203 generates an MMW signal based on the converted analog signal. The transmission antenna 204 transmits the MMW signal generated by the MMW signal generation unit 203 in the direction of an object to be measured. The reception antennas 205 and 206 receive a signal obtained by reflecting and scattering the signal transmitted from the transmission antenna 204 onto the object to be measured. The reception unit 207 converts the signal received by the reception antenna into a baseband electrical signal. The A/D conversion unit 208 converts the analog reception signal processed by the reception unit 207 into a digital signal.

If a control signal such as a transmission or reception instruction is inputted from a central control unit for controlling the entire apparatus, the control unit 201 controls the entire operation of the millimeter-wave transmission/reception units for generating millimeter-wave signals or processing reception signals. That is, the control unit 201 controls each component according to a transmission or reception mode. Particularly, the control unit 201 performs a control for ensuring switching of the transmission and reception antennas. The control unit 201 also provides energy necessary for generating a millimeter-wave signal to the MMW signal generation unit 203.

The MMW signal generation unit 203 receives the control signal and energy, generated by the control unit 201, from the D/A conversion unit 202 and generates a millimeter-wave signal to be transmitted to the object to be measured. In this case, the transmission signal generated by the MMW signal generation unit 203 is frequency-modulated and amplified, and then transmitted through the transmission antenna 204.

The millimeter-wave signal generated by the MMW signal generation unit 203 is transmitted to the object to be measured through the transmission antenna 204.

The transmission signal transmitted to the object to be measured is reflected and scattered by the object to be measured, and the reception antennas 205 and 206 in each of the plurality of millimeter-wave transmission/reception units receive the reflected and scattered reception signal. In the present invention, the reception antenna may be configured with one antenna or may be configured with two or more antenna so as to improve reception performance.

The reception unit 207 down-converts the millimeter-wave signal received by the one or more reception antennas 205 and 206 into a baseband reception signal and provides the converted analog signal to the A/D conversion unit 208.

The A/D conversion unit 208 converts the baseband analog signal inputted from the reception unit 207 into a digital signal and provides the converted digital signal to the control unit 201.

The control unit 201 processes the reception signal inputted from the A/D conversion unit 208, and provides the processed reception signal to the digital signal processing unit 106.

FIG. 3 is a block configuration diagram illustrating another embodiment of the millimeter-wave transmission/reception unit in accordance with the present invention. The millimeter-wave transmission/reception unit in accordance with the embodiment of the present invention is configured by adding a camera 301 and an image signal conversion unit 302 to the configuration of the millimeter-wave transmission/reception unit illustrated in FIG. 2.

The millimeter-wave transmission/reception unit illustrated in FIG. 3 further includes a camera 301 and an image signal conversion unit 302. The camera 301 directly obtains an image of the object to be measured. The image signal conversion unit 302 converts an image signal of the object to be measured, obtained by the camera 301, into a digital signal of a specific format to be used in the digital signal processing unit, and provides the converted digital signal to the control unit 302.

The digital signal processing unit 106 performs correction of the sizes of the human body by comparing information on an actual image of the object to be measured with data on the object to be measured, obtained using the millimeter-wave signal.

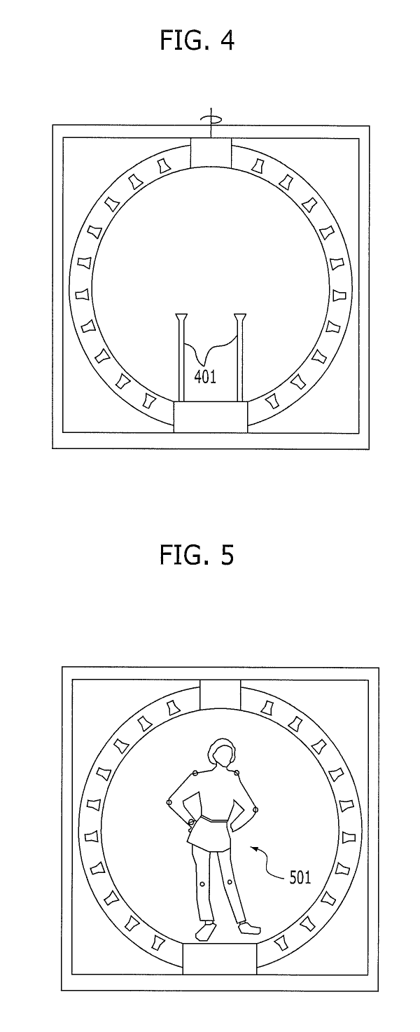

FIG. 4 illustrates an apparatus for measuring sizes of a human body using millimeter waves in accordance with another embodiment of the present invention.

Referring to FIG. 4, when sizes of a human body are measured, a user must maintain a stopped state for a measurement time (10 seconds or so), but a measurement error may occur due to a slight movement of the user. In order to prevent the measurement error, an auxiliary handle 401 for enabling the user to fix its posture is provided near the support platform on which the user stands as an object to be measured. The auxiliary handle 401 functions to help the user maintain a stopped posture for the measurement time.

FIG. 5 illustrates a method for measuring sizes of a human body using landmarks in accordance with an embodiment of the present invention.

The landmark may be made of a metal or radio wave absorber. The metal has a property of totally reflecting a millimeter-wave signal. The radio wave absorber has a property of absorbing a radio wave with a specific frequency. The plurality of landmarks 50 made of the metal or radio wave absorber are attached to an object to be measured, using the property of the metal or radio wave absorber. Accordingly, the landmarks can be used to correct sizes of a human body when the digital signal processing unit 106 extracts the sizes of the human body using data measured based on the landmark as a reference point.

Although only the usage for measuring sizes of a human body has been described in the embodiment of the present invention, it will be obvious that the present invention may be applied to security and medical systems having the same configuration as the present invention.

In accordance with the exemplary embodiments of the present invention, sizes of a human body can be measured even in the state that a user puts on simple clothes, using a characteristic in which a signal of a millimeter-wave signal does not pass through the human body but passes through the clothes, thereby improving user's convenience. Further, the measuring apparatus having a circular structure is implemented, so that it is possible to facilitate implementing a 3D anatomical model using the spherical coordinate system. Furthermore, a landmark made of metal is used as a reference point for measurement using the reflection property of the metal in the millimeter-wave band, so that it is possible to improve accuracy in measuring sizes of a human body.

The above-described methods can also be embodied as computer programs. Codes and code segments constituting the programs may be easily construed by computer programmers skilled in the art to which the invention pertains. Furthermore, the created programs may be stored in computer-readable recording media or data storage media and may be read out and executed by the computers. Examples of the computer-readable recording media include any computer-readable recoding media, e.g., intangible media such as carrier waves, as well as tangible media such as CD or DVD.

While the present invention has been described with respect to the specific embodiments, it will be apparent to those skilled in the art that various changes and modifications may be made without departing from the spirit and scope of the invention as defined in the following claims.

What is claimed is:

1. A measuring apparatus using millimeter waves, the apparatus comprising:
a plurality of millimeter-wave transmission/reception units configured to be arranged in a circular shape around an object to be measured, transmit a millimeter wave to the object to be measured, and receive a signal reflected or scattered by the object to be measured;
a rotation unit configured to rotate a circular structure in which the plurality of millimeter-wave transmission/reception units are arranged; and
a digital signal processing unit configured to analyze the object to be measured using size and phase information of the signal received by the plurality of millimeter-wave transmission/reception units,
wherein, in one measurement cycle in which any one of the plurality of millimeter-wave transmission/reception units operates in a transmission mode and all the millimeter-wave transmission/reception units operate in a reception mode, the measurement is sequentially performed until when all the millimeter-wave transmission/reception units operate one by one in the transmission mode.

2. The apparatus of claim 1, wherein, when the one measurement cycle is completed, the circular structure is rotated to a predetermined angle by the rotation unit, and another measurement cycle is then performed.

3. The apparatus of claim 2, wherein the millimeter-wave transmission/reception unit comprises:
a control unit configured to control the transmission and reception modes, control the generation of the millimeter wave, and provide a measured result to the digital signal processing unit;
a millimeter-wave signal generation unit configured to generate a millimeter-wave signal under a control of the control unit;
a transmission antenna configured to transmit, to the object to be measured, the millimeter-wave signal generated by the millimeter-wave signal generation unit;
a reception antenna configured to receive the signal reflected or scattered by the object to be measured; and
a reception unit configured to process the signal inputted from the reception antenna and provide the processed signal to the control unit.

4. The apparatus of claim 3, wherein the millimeter-wave transmission/reception unit further comprises:
an image photographing unit configured to photograph an image of the object to be measured; and
an image signal conversion unit configured to process the image photographed by the image photographing unit.

5. The apparatus of claim 4, wherein the digital signal processing unit corrects the measured result of the object to be measured based on the photographed image processed by the image signal conversion unit.

6. The apparatus of claim 3, wherein the reception antenna is configured with two or more reception antennas so as to improve reception performance.

7. The apparatus of claim 3, wherein, when the measurement in directions of 360 degrees is completed through the millimeter-wave transmission/reception units, the digital signal processing unit estimates sizes of a human body of the object to be measured using an algorithm for solving an inverse scattering equation, based on the measured result.

8. The apparatus of claim 7, further comprising a support platform configured to support the object to be measured.

9. The apparatus of claim 8, further comprising an auxiliary handle disposed near the support platform so as to prevent a movement of the object to be measured.

10. The apparatus of claim 7, further comprising a plurality of landmarks made of a material totally reflecting or absorbing a millimeter wave of the signal transmitted by the millimeter-wave transmission/reception unit and attached to the object to be measured as measurement reference points.
